(19) United States
(12) Patent Application Publication
(10) Pub. No.: US 2023/0108730 A1
(43) Pub. Date: Apr. 6, 2023

(54) BIENZYMATIC BIOSENSOR FOR THE DETECTION OF LACTULOSE IN BLOOD

(71) Applicant: TRIVIUMVET DESIGNATED ACTIVITY COMPANY, Waterford (IE)

(72) Inventors: Louise GRUBB, County Waterford (IE); Stuart FITZGERALD, County Waterford (IE); Liam BYRNE, County Waterford (IE)

(73) Assignee: TRIVIUMVET DESIGNATED ACTIVITY COMPANY, Waterford (IE)

(21) Appl. No.: 17/904,684

(22) PCT Filed: Feb. 17, 2021
(86) PCT No.: PCT/EP2021/053923

§ 371 Date: Aug. 19, 2022

(30) Foreign Application Priority Data
Feb. 20, 2020 (EP) 20158486.9
Oct. 1, 2020 (EP) 20199717.8

(51) Int. Cl. G01N 33/53; G01N 33/68

(52) U.S. Cl. CPC G01N 33/5302; G01N 33/68; G01N 2800/062

(57) ABSTRACT

A method for determining the concentration of lactulose in a blood sample comprises the steps of converting lactulose in the blood sample into an entity including galactose, and converting the galactose into D-galacto-hexodialdose and hydrogen peroxide, which is detected. The detected quantity is processed to generate an output which is representative of the concentration of lactulose in the blood sample.

### INTRODUCTION

The invention relates to an assay for diagnosing gastric ulceration in equines, canines and other animals.

Gastric ulcer syndrome is an important cause of morbidity in equines, canines, and humans. 76% of elite event horses have gastric ulceration, between 98-100% of racehorses in training are effected and 50% of leisure horses also suffer from the problem.

https://www.horseandhound.co.uk/horse-care/vet-advice/gastric-ulcers-dispelling-the-myths-282000#2epgJ1skq0AkahmY.99

Similarly, up to 75% of dogs receiving long-term nonsteroidal anti-inflammatory drug (NSAID) treatment have been reported to suffer from gastric ulceration. (https://onlinelibrary.wiley.com/doi/10.1111/jvim.16057)

The only definitive diagnostic test available is endoscopy which requires general anaesthetic or sedation of the subject and visualisation of the gastric mucosa by an experienced veterinarian or doctor. The availability of a veterinarian or doctor to carry out the procedure, the risk of a general anaesthetic/sedation and the cost of the procedure make it prohibitive for use as a diagnostic tool in general practice. It is common practice for veterinarians and doctors to prescribe treatments based on clinical presentation only.

Therefore, the development of an inexpensive screening test at the point-of-care would ensure early diagnosis and more accurate treatment of gastric ulceration. Avoiding the need for sedation or general anaesthesia in diagnosing gastric ulceration would eliminate risks such as adverse cardiac and neurological effects of sedative and anaesthetic drugs.

CN105784868A describes a method using ion chromatography with an amperometric detector to detect lactulose in milk. Cox et al. Clinica Chimica Acta 263 (1997) 197-205 describes a method to detect mannitol and lactulose in a sample of deproteinated blood. The ratio of lactulose to mannitol is used as an indicator of coeliac disease. The method uses liquid chromatography with electrochemical detection and a high-performance anion-exchange column with an associated guard column. Similarly, Sørensen et al. Clinica Chimica Acta 264 (1997)103-115 describes HPLC with pulsed amperometry to assess levels of sugar probes in healthy dogs. Rodriguez et al.

The Canadian Journal of Veterinary Research 73 (2009) 217-273 used gas chromatography — mass spectrometry to assess intestinal permeability of a mixture of five sugar probes to in healthy dogs.

### SUMMARY

According to the invention there is provided a method for determining the concentration of lactulose in a blood sample comprising the steps of:

a) converting lactulose in the blood sample into an entity including galactose;
b) converting the galactose into D-galacto-hexodialdose and hydrogen peroxide in the presence of oxygen or a redox mediator;
c) detecting one or more of the following which are provided by step (b):
quantity of hydrogen peroxide, and/or
quantity of D-galacto-hexodialdose, and/or
a change in the quantity of redox mediator, and/or
a change in the quantity of oxygen; and
   d) processing the detected quantity or change in quantity provided by step (c) to generate an output which is representative of the concentration of lactulose in the blood sample.

In one case the step (a) conversion of lactulose in the blood sample into galactose is an enzymatic conversion. The enzyme for converting lactulose in the blood sample into galactose may be β-galactosidase.

In one case the step (b) conversion of the galactose into D-galacto-hexodialdose and hydrogen peroxide is an enzymatic conversion. In one case the step (b) enzyme for converting the galactose into D-galacto-hexodialdose and hydrogen peroxide is galactose oxidase.

In one embodiment the quantity of hydrogen peroxide is detected in step (c) and processed in step (d) to generate the output.

In one case an electrochemical biosensor including a working electrode, a counter electrode and a reference electrode is provided, at least one enzyme is immobilized on the working electrode surface, the step (a) and (b) conversions are performed by contact with the immobilized enzyme or enzymes when the working electrode is exposed to the sample and a voltage is applied across the electrodes.

The working electrode may include platinum (Pt) and the enzymes are drop cast onto the working electrode surface using a cross-linker which may comprise glutaraldehyde. In one case there are at least two immobilized enzymes including β-galactosidase for said step (a) conversion and galactose oxidase for said step (b) conversion.

In one case the hydrogen peroxide is oxidised on the working electrode surface in step (b). The hydrogen peroxide may be detected in step (c), and said detection is by detection of the current flow in the sensor.

In some cases, a redox mediator is used as an electron transporter between the enzyme(s) and the working electrode. If so, a change in the quantity of the redox mediator as a result of the conversion of the galactose into D-galacto-hexodialdose and hydrogen peroxide may be detected and processed to generate the output. The redox mediator may, for example, be selected from ferrocene and its derivatives, quinone-based compounds, ferro/ferricyanide, or various redox organic polymers or inorganic complexes or combinations thereof, and it may comprise osmium.

The blood sample may be a sample of blood serum or blood plasma.

The blood sample may be from a human or a non-human animal, such as an equine or a canine.

The invention also provides an apparatus for determining the concentration of lactulose in a blood sample, the apparatus comprising:
a converter for:
   converting lactulose in the blood sample into an entity including galactose; and
   converting the galactose into D-galacto-hexodialdose and hydrogen peroxide in the presence of oxygen or a redox mediator;
a detector for:
   detecting one or more of the following which are provided by the converter:
      quantity of hydrogen peroxide, and/or
      quantity of D-galacto-hexodialdose, and/or
      a change in the quantity of redox mediator, and/or
      a change in the quantity of oxygen; and
a processor for processing the detected quantity or change in quantity provided by the detector to generate an output which is representative of the concentration of lactulose in the blood sample.

The blood sample may be a sample of blood serum or blood plasma.

In one case the converter comprises a substrate for an enzyme to perform conversion of lactulose in the blood sample into galactose. The enzyme for converting lactulose in the blood sample into galactose may be β-galactosidase.

In one case the converter comprises a substrate for an enzyme for conversion of the galactose into D-galacto-hexodialdose and hydrogen peroxide. The enzyme for converting the galactose into D-galacto-hexodialdose and hydrogen peroxide may be galactose oxidase.

In one case the detector is adapted to detect a quantity of hydrogen peroxide, and the processor is adapted to generate the output according to the detected quantity of hydrogen peroxide.

In one embodiment the converter and the detector are integrated in an electrochemical biosensor including a working electrode, a counter electrode and a reference electrode, at least one enzyme is immobilized on the working electrode surface and the converter and the detector are adapted to perform the conversions by contact with the immobilized enzyme or enzymes when the working electrode is exposed to the sample, and a driver to apply a voltage across the electrodes.

In one case the working electrode includes Pt and the enzymes are drop cast onto the working electrode surface using a cross-linker which may comprise glutaraldehyde. There may be at least two immobilized enzymes including β-galactosidase and galactose oxidase.

In one case the voltage drive is operable to cause hydrogen peroxide to be oxidised on the working electrode surface, and the detector is adapted to measure current flow arising from said applied drive.

The invention also provides a method for diagnosing gastric ulceration in a subject comprising the steps of:—
administering lactulose to the subject;
taking a sample of blood from the subject after administering lactulose; and
in vitro determining the concentration of lactulose in the blood sample as an indicator of gastric ulceration.

The blood sample may be a sample of blood serum or blood plasma. In one case the method comprises the step of converting the blood sample into blood plasma and determining the concentration of lactulose in the blood plasma. In one case the conversion is achieved by centrifugation following collection of the blood sample into anticoagulant-coated tubes.

The use of anticoagulant-coated tubes prior to centrifugation of blood samples yielded liquid plasma suitable for analysis. In some cases, the presence of small concentrations of lactulose in blood, were found to result in the formation of a serum gel when blood samples were collected into plain serum tubes. The anticoagulant in one case is lithium heparin. Other anticoagulants such as sodium citrate, sodium fluoride or potassium oxalate may also have been used for this purpose.

In one embodiment a determined concentration of more than 100 micromolar lactulose in a blood plasma sample within 60 minutes of administration is indicative of gastric ulceration.

In some embodiments the lactulose is administered in an amount of from 0.5 to 1.0 gram per kg bodyweight of the subject. In some cases, the lactulose is administered in an amount of from 0.6 to 0.7 gram per kg bodyweight of the subject.

The volume and concentration of the lactulose solution are selected to determine the optimum volume and osmotic gradient given the likely anatomical distribution and extent of gastric ulcers, and the dimensions of the fasted stomach.

The method may comprise the step of waiting for a period of time after administering lactulose before taking a blood sample, the waiting period after administering lactulose and before taking a blood sample may be from 5 to 100 minutes, or 50 to 100 minutes.

In some cases, the lactulose is administered after a fasting period, the fasting period may be from 6 to 24 hours, or 6 to 12 hours.

In one embodiment the method comprises converting lactulose in the blood sample into an entity including galactose and processing the galactose to generate an output which is representative of the concentration of lactulose in the blood sample.

In one case said processing comprises converting the galactose into D-galacto-hexodialdose and hydrogen peroxide in the presence of oxygen or a redox mediator and detecting one or more of the following which are provided:
quantity of hydrogen peroxide, and/or
quantity of D-galacto-hexodialdose, and/or
a change in the quantity of redox mediator, and/or
a change in the quantity of oxygen; and
processing the detected quantity or change in quantity to generate an output which is representative of the concentration of lactulose in the blood sample.

The method may comprise converting the galactose into D-galacto-hexodialdose and hydrogen peroxide, and detecting one or more of the quantity of hydrogen peroxide, and/or the quantity of D-galacto-hexodialdose.

In one case the conversion of lactulose in the blood sample into galactose is an enzymatic conversion. The enzyme for converting lactulose in the blood sample into galactose may be β-galactosidase. In one case the conversion of the galactose into D-galacto-hexodialdose and hydrogen peroxide is an enzymatic conversion. The enzyme for converting the galactose into D-galacto-hexodialdose and hydrogen peroxide may be galactose oxidase. The quantity of hydrogen peroxide may be detected and processed to generate the output.

In one embodiment an electrochemical biosensor with a working electrode and a reference electrode is provided, at least one enzyme is immobilized on the working electrode surface and the conversion(s) are performed by contact with the immobilized enzyme or enzymes when the working electrode is exposed to the sample and a voltage is applied across the electrodes.

The working electrode may include Pt and the enzyme(s) are drop cast onto the working electrode surface using a cross-linker which may comprises glutaraldehyde. In one case there are at least two immobilized enzymes including β-galactosidase and galactose oxidase.

In one case hydrogen peroxide is oxidised on the working electrode surface. The hydrogen peroxide may be detected and said detection is by detection of the current flow in the sensor. In one case a change in the quantity of the redox mediator as a result of the conversion of the galactose into D-galacto-hexodialdose and hydrogen peroxide is detected and processed to generate the output.

In some cases, a redox mediator is used as an electron transporter between the enzyme(s) and the working electrode. If so, change in the quantity of the redox mediator as a result of the conversion of the galactose into D-galacto-hexodialdose and hydrogen peroxide may be detected and processed to generate the output. The redox mediator may, for example, be selected from ferrocene and its derivatives, quinone-based compounds, ferro/ferricyanide, or various redox organic polymers or inorganic complexes or combinations thereof, and the redox mediator may comprise osmium.

To simplify the assay, lactulose is preferably the only sugar administered to the subject as part of the diagnostic test.

The blood sample may be is from a human or a non-human animal such as an equine or a canine.

According to the invention there is provided a method for determining the concentration of lactulose in a blood sample comprising the steps of:
converting lactulose in the blood sample into galactose;
converting galactose into D-galacto-hexodialdose and hydrogen peroxide; and
measuring the concentration of D-galacto-hexodialdose and/or hydrogen peroxide, which is proportional to the concentration of lactulose in the blood sample.

In one case converting lactulose in the blood sample into galactose and converting galactose into D-galacto-hexodialdose and hydrogen peroxide is carried out by an enzyme system. The step of measuring the concentration of D-galacto-hexodialdose and/or hydrogen peroxide may be carried out by an electrode such as a catalytic electrode.

In one case the converting and measuring steps are carried out by a sensor having an enzyme system comprising β-galactosidase for converting lactulose into galactose and fructose and galactose oxidase for converting galactose into D-galacto-hexodialdose and hydrogen peroxide and a catalytic electrode.

The sensor may comprise an electrode layer and an enzyme layer. The enzyme layer may comprise galactose oxidase. The enzyme layer may comprise β-galactosidase.

In one case the method comprises the step of converting the signal produced from the electrode into an output indicative of the concentration of lactulose in the blood sample.

The step of converting the signal produced from the electrode into an output indicative of the concentration of lactulose in the blood sample may be carried out by an instrument.

The instrument may be a hand-held instrument.

In one case the blood sample is from a human.

In another case the blood sample is from a non-human animal, particularly an equine or a canine.

The invention also provides an electrochemical assay for determining the concentration of lactulose in a blood comprising:
a sensor having an enzyme system comprising β-galactosidase for converting lactulose into galactose and fructose and galactose oxidase for converting galactose into D-galacto-hexodialdose and hydrogen peroxide;
a catalytic electrode; and
an instrument for converting the signal produced from the electrode into an output indicative of the concentration of lactulose in the blood sample.

In one case the sensor comprises an electrode layer and an enzyme layer.

The enzyme layer may comprise galactose oxidase. In one case the enzyme layer comprises β-galactosidase.

In one case the instrument is a hand-held instrument.

In one case the blood sample is from a human.

In another case the blood sample is from a non-human animal, particularly an equine or a canine.

The invention also provides use of an electrochemical assay for determining the concentration of lactulose in a blood sample, the assay comprising:
a sensor having an enzyme system comprising: β-galactosidase for converting lactulose into galactose and fructose; and galactose oxidase for converting galactose into D-galacto-hexodialdose and hydrogen peroxide;
a catalytic electrode; and
an instrument for converting the signal produced from the electrode into an output indicative of the concentration of lactulose in a sample of blood.

In one case the sensor comprises an electrode layer and an enzyme layer.

The enzyme layer may comprise galactose oxidase. The enzyme layer may comprise β-galactosidase.

The instrument may be a hand-held instrument.

The invention also provides a method for diagnosing gastric ulceration in a subject comprising the steps of
administering lactulose to the subject;
taking a sample of blood from the subject after administering lactulose; and
determining the concentration of lactulose in the blood sample as an indicator of gastric ulceration.

In one case the method comprises the step of waiting for a period of time after administering lactulose before taking a blood sample. In one case the waiting period after administering lactulose before taking a blood sample is from 50 to 100 minutes.

In one case the lactulose is administered after a fasting period. Typically, the fasting period is from 6 to 12 hours.

The subject may be a human or a non-human animal. In one case the subject is an equine. In another case the subject is a canine.

The method may comprise:
converting lactulose in the blood sample into galactose;
converting galactose into D-galacto-hexodialdose and hydrogen peroxide; and
measuring the concentration of D-galactohexodialdose and/or hydrogen peroxide, which is proportional to the concentration of lactulose in the sample.

### BRIEF DESCRIPTION OF THE DRAWINGS

The invention will be more clearly understood from the following description thereof, given by way of example only, in which:

FIG. 1 is a diagram of a diagnostic system of the invention;

FIG. 2 is a diagram of a sensing system;

FIG. 3 is a diagram illustrating the operation of the biosensor;

FIG. 4 is a graph of the concentration of lactulose detected in plasma by severity of ulceration; and

FIG. 5 is an amperometric i-t plot for the quantification of lactulose in a canine plasma sample.

### DETAILED DESCRIPTION

Lactulose is not found naturally in the mammalian circulation, as it is nonabsorbable and indigestible. We have surprisingly found that the large lactulose molecule passes much more readily into the bloodstream across ulcerated gastric tissue than through “leaky” tight junctions between intestinal cells as are seen in immunologic disease of the intestinal tract such as coeliac disease. Direct exposure of underlying submucosal tissues in the gastric wall to lactulose facilitates far greater uptake of the molecule than is possible through a porous, but intact mucosal layer.

The invention provides a point of care test device to measure lactulose in blood at micromolar concentrations that is used in the diagnosis of gastric ulcers.

We have found that plasma lactulose levels in excess of 100 μM within 60 minutes of oral administration of 0.6 to 0.7 g/kg lactulose in a 20% w/v solution may be used to diagnose gastric ulceration in monogastric animals. More generally, plasma lactulose levels in excess of 100 μM within 60 minutes of oral administration of 0.5 to 1.0 g/kg lactulose indicate the presence of gastric ulceration. Several treatments that may be used after detection of gastric ulceration as described herein are outlined in detail below.

An apparatus for performing the test comprises a catalytic electrode and an instrument for converting the signal produced from the electrode into an output indicative of the concentration of lactulose in the blood sample. In one case the instrument comprises a handheld portable lactulose meter, which includes an electrochemical lactulose sensor, having a sensor output related to lactulose in a blood sample. Contained within this portable device are electrochemical sensor(s), LCD screen, a battery compartment and various buttons to operate the device, e.g. on/off or to take a reading. We provide an electrochemical method for measuring lactulose in blood at the required micro molar concentrations.

One assay of the invention uses two enzymes—β-galactosidase and galactose oxidase. The β-galactosidase converts the lactulose to galactose and fructose. Using appropriate fasting requirements, for example 6 to 12 hours, it is ensured that there is negligible galactose present in circulation from other dietary sources. The galactose can now be uniquely measured from the lactulose breakdown, without any concern for interference from normal circulating levels. Such electrochemical sensors can be manufactured in a manner similar to glucose test strips, and for the system to operate in a very similar way to taking a blood glucose measurement.

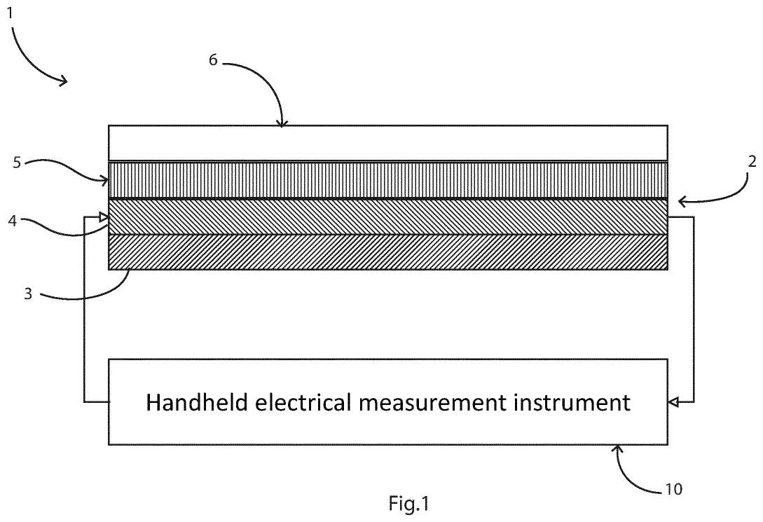

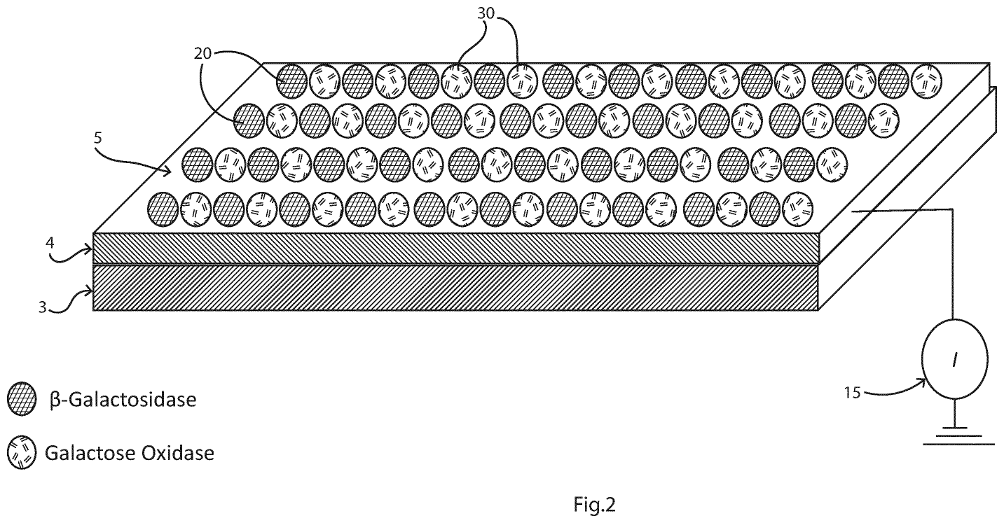

Referring to FIGS. 1 to 3, a biosensor 1 for implementing the method in one example is illustrated. It has a catalytic electrode and an instrument for converting the signal produced from the electrode into an output indicative of lactulose in a sample of blood. The biosensor 1 comprises a catalytic electrode assembly 2 with, in order from the lower end, a flexible substrate 3, a printed working electrode 4, and a layer 5 of immobilized enzymes on the surface of the working electrode 4. This diagram also shows a blood sample layer 6 for illustrative purposes. The electrode drive or instrument is indicated generally by the numeral 10, and FIG. 2 shows a current source 15 which is part of it. A reference electrode is also incorporated in 10, as is known in the art.

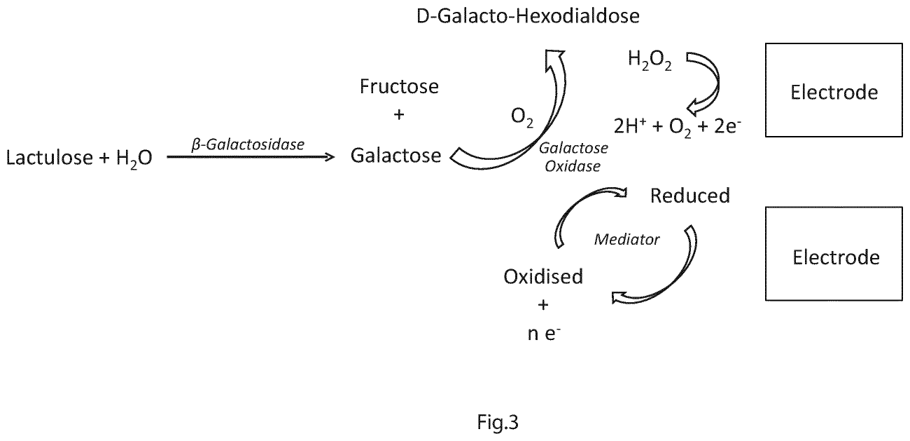

As shown in FIG. 2 the enzymes are β-galactosidase 20 and galactose oxidase 30. For illustrative purposes they are shown as an array with alternate species, but the pattern may be different. In the biosensor 1 the electrodes 2 are driven by the drive 10 to perform the functions of a converter for converting lactulose in the blood sample 6 into an entity including galactose, and also converting the galactose into D-galacto-hexodialdose and hydrogen peroxide in the presence of oxygen or a redox mediator. This is illustrated graphically in FIG. 3. The biosensor 1 also functions as a detector for detecting one or more of the following which are provided by the converter: quantity of hydrogen peroxide, and/or quantity of D-galacto-hexodialdose. The drive 10 includes a processor for processing the detected quantity or change in quantity provided by the detection to generate an output which is representative of the concentration of lactulose in the blood sample.

Advantageously, the biosensor 1 provides the substrate 4 for the β-galactosidase enzyme to perform conversion of lactulose in the blood sample into galactose, and also the enzyme galactose oxidase enzyme for converting the galactose into hydrogen peroxide H₂O₂. Also, the biosensor functions as a detector to detect the quantity of hydrogen peroxide, and the processor generating the output representative of lactulose in the blood sample according to the detected quantity of hydrogen peroxide.

The working electrode 5 is in the form of a 2mm diameter Pt disc and the enzymes are drop cast onto the working electrode surface using a cross-linker. Also, the cross-linker comprises glutaraldehyde. The voltage driver 10 causes hydrogen peroxide to be oxidised on the working electrode 4 surface, and measures current flow arising from said applied drive.

It is advantageous that the converter and the detector are integrated in the electrochemical biosensor 1 with the working electrode 4 and the reference electrode, the enzymes are immobilized on the working electrode surface and perform the conversions by contact of the sample with the immobilized enzymes when the electrodes are immersed in the sample and the drive 10 applies a voltage across the electrodes.

The electrochemical biosensor 1 performs indirect detection of lactulose in buffer and plasma samples using the electrocatalytic surface 4/5 as the working electrode, being modified with the dual enzyme system comprising β-galactosidase (β-Gal) and galactose oxidase (GalOx), these enzymes being immobilised using a cross-linker.

In one case, a 20 μL mix of β-Gal/GalOx/Glutaraldehyde (4 units /13.3 units /0.5 wt. %) was prepared in Phosphate-buffered saline (PBS) solution 0.1 M pH 7.3. 5 μL of the mix was then drop-casted at the working electrode 4 surface and left to dry at room temperature.

The electrochemical biosensor was then formed by use of a 3-electrode system of the working electrode (the dual enzyme-modified Pt electrode 4/5), a Silver/Silver Chloride (Ag/AgCl) reference electrode, and a Pt wire as auxiliary electrode.

The three electrodes were immersed in a known volume of buffer solution 6 to form the electrochemical cell and an oxidative potential of +600 mV was applied by the drive 10 between the working and reference electrodes. The current generated was allowed to reach a steady state. When the current had reached steady state, the sample 6 containing lactulose was added to the buffer solution and the current response monitored. Sample consisted of a volume of a standard lactulose concentrations or unknown concentrations of lactulose in buffer or plasma.

A faradaic oxidative or reductive current is generated in response to the oxidation of reduction of hydrogen peroxide (or redox mediator or oxygen). The current signal is proportional to the concentration of lactulose added to the cell. The current is processed in such a way as to measure the magnitude of change of the current signal, the charge passed over time or the rate of change of the current over time in response to the lactulose added to the cell.

In one case, the biosensor operation is based on the detection of oxygen (principle of the Clark electrode) where the working electrode is functionalised with the dual enzyme system. Oxygen oxidation or reduction can be monitored by a change in current, which will be dependent on enzyme activity which, in turn, depends on lactulose concentration added to the cell.

Another example is biosensor detection of an enzymatic product of the reaction cycle of lactulose with the immobilised enzyme(s). For example, the product may be hydrogen peroxide or fructose or galactose.

The enzymatic generation of hydrogen peroxide and its subsequent oxidation or reduction at the electrode surface can be monitored by a change in current which will be dependent on enzyme activity which, in turn, depends on lactulose concentration added to the cell.

In the case of fructose or galactose these sugars are detected enzymatically or via a ligand binding interaction at the electrode surface. For example, the sugars may be catalytically converted to electroactive products such as hydrogen peroxide that can be oxidised or reduced at the electrode surface. Alternatively, ligand-based binding of sugars to the surface of the electrode can be monitored electrochemically (using a bulk redox mediator) or via a capacitive change.

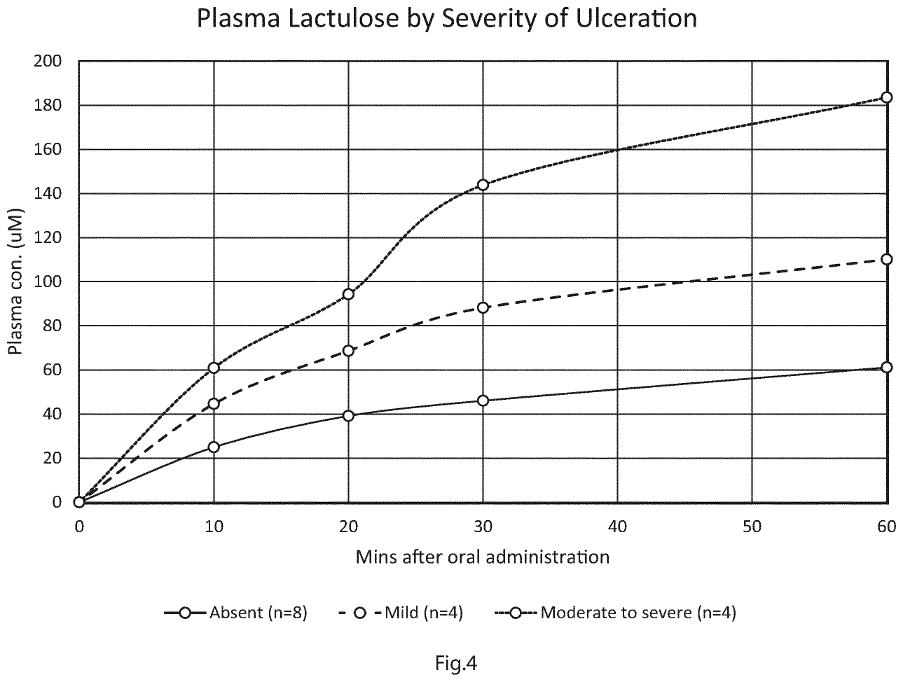

FIG. 4 is a graph of the concentration of lactulose detected in plasma by severity of ulceration. The biosensor determines lactulose concentration in the blood plasma after oral administration, and the concentration in the plasma increases as illustrated with time duration after the oral administration of lactulose to the canine.

In more general terms the biosensor 1 performs a method for determining the concentration of lactulose in a blood sample comprising the steps of:

(a) converting lactulose in the blood sample into an entity including galactose;
(b) converting the galactose into D-galacto-hexodialdose and hydrogen peroxide in the presence of oxygen or a redox mediator;
(c) detecting quantity of hydrogen peroxide, and
(d) processing the detected quantity or change in quantity provided by step (c) to generate an output which is representative of the concentration of lactulose in the blood sample.

The step (a) conversion of lactulose in the blood sample into galactose is an enzymatic conversion. The enzyme for converting lactulose in the blood sample galactose may advantageously be β-galactosidase. The step (b) conversion of the galactose into D-galacto-hexodialdose and hydrogen peroxide is an enzymatic conversion. The step (b) enzyme for converting the galactose into D-galacto-hexodialdose and hydrogen peroxide is galactose oxidase. The quantity of hydrogen peroxide is detected in step (c) and processed in step (d) to generate the output.

Hence, the step (a) and (b) conversions are performed by contact with the immobilized enzymes when the electrodes are immersed in the sample and a voltage is applied across the electrodes. The hydrogen peroxide is oxidised on the working electrode surface in step (b), and its detection is by detection of the current flow in the sensor.

### Redox Mediator Alternative

If a redox mediator is added the sensor may function to determine a change in the quantity of the mediator and/or a change in the quantity of oxygen.

The mediator is used as an electron transporter between the enzyme(s) and the working electrode. If so, change in the quantity of the redox mediator as a result of the conversion of the galactose into D-galacto-hexodialdose and hydrogen peroxide may be detected and processed to generate the output. The redox mediator may, for example, be selected from ferrocene and its derivatives, quinone-based compounds, ferro/ferricyanide, or various redox organic polymers or inorganic complexes or combinations thereof, and may comprise osmium.

A blood sample is placed on a disposable sensing electrode, such as a printed/flexible electrochemical enzyme sensing electrode. The blood sample may uniquely contain lactulose from exogenous sources as a result of the presence of an ulcer. Lactulose is converted to galactose and fructose by the presence of the enzyme β-galactosidase, which is immobilised, or deposited onto the electrochemical sensing electrodes. Endogenous galactose can be eliminated by pre-test fasting practices. Any galactose will thus be uniquely present due to the presence of exogenous lactulose in the sample. Galactose can be detected at a suitably catalytic electrode at which galactose oxidase has been immobilised (such as described by Kanyong et al Microchim Acta 2017, 184, 3663-3671), and which is capable of measuring and quantifying galactose within the range of zero to several hundred micromolar, which is proportional to the concentration of lactulose in the blood sample.

The time for fasting, lactulose bolus administration, timing of blood sampling and handling are optimised.

### EXAMPLE 1

An animal is fasted for a specific period of time after which a lactulose gel is administered. After a specific period of time (typically 50-100 minutes later), circulating blood is collected and tested as described above. A typical lactulose bolus contains 0.5 to 1 g lactulose per kg body weight at a concentration of a 10% solution. For example, a 250 g sachet of lactulose is diluted with 2.25 L water and delivered by a veterinarian via a nasal gastric tube. In another example, 15 mL of a 66.7% w/v lactulose syrup may be diluted with 40 mL of water.

### EXAMPLE 2-CLINICAL INVESTIGATION (FIG. 4)

A model of canine gastric ulceration was developed using a combination of orally administered nonsteroidal anti-inflammatory drugs (NSAIDs) at doses appropriate to induce ulceration without concomitant renal or hepatic dysfunction. Healthy laboratory dogs (14.0 to 16.7 kg) were screened to exclude individuals with clinically silent disorders, including gastrointestinal disease. Under the GCP study protocol, each dog was administered 50 ml of a 20% w/v lactulose solution orally (0.6-0.7 g/kg lactulose PO) following a 24-h fast at baseline and following a period of ulcer induction. On each occasion, peripheral venous blood was drawn from each dog into 1.3-ml Sarstedt blood tubes containing lithium heparin as an anticoagulant at 10, 20, 30 and 60 minutes following bolus administration of the lactulose solution. The Sarstedt tube product number was 41.1393.005. Blood samples were centrifuged for 15 minutes at 3000 rpm, before decanting plasma into plain tubes; plasma was stored at −80° C. until analysis. Prior to lactulose administration, endoscopy was performed on each dog, and the presence and extent of gastric ulceration were assessed by a blinded specialist veterinary gastroenterologist. Following confirmation of the presence of ulceration, NSAID administration was discontinued and post ulcer induction lactulose administration and plasma collection were performed.

The use of an anticoagulant was necessitated by the tendency of serum to congeal following lactulose administration. The presence of micromolar concentrations of lactulose in blood, consistent with levels expected to be achieved in canine and other patients with gastric ulceration were found to result in the formation of a serum gel when blood samples were collected into plain serum tubes. The viscosity of the serum gel so formed prevented dilution and analysis of these blood samples. The use of lithium heparin anticoagulant-coated tubes prior to centrifugation of blood samples yielded liquid plasma suitable for analysis. Other anticoagulants such as sodium citrate, sodium fluoride or potassium oxalate may also have been used for this purpose.

The dogs used in the study were subsequently treated with 0.5 to 1.0 mg/kg omeprazole twice daily for 3 weeks. As stated by the American College of Veterinary Internal Medicine's expert panel (Marks et al. “ACVIM consensus statement: support for rational administration of gastrointestinal protectants to dogs and cats.”Journal of veterinary internal medicine 32.6 (2018): 1823-1840) “proton pump inhibitors administered twice daily are superior to other gastroprotectants for treating acid-related gastroduodenal ulceration and erosion” in dogs and cats. Furthermore, study dogs were transitioned onto an appropriate low-fibre, low-fat, high-moisture diet. Soft foods are typically recommended for canine gastric ulcer patients in order to minimize mechanical trauma to the stomach; lower fibre content is associated with greater calorie density to aid in meeting nutritional requirements, while the lower fat content promotes rapid gastric emptying. Symptoms of gastric ulceration (including vomiting, inappetence, abdominal pain, melaena and diarrhea), present in all study dogs prior to initiation of omeprazole treatment, resolved within several days in each case. Laboratory evidence of gastric ulceration (including anemia, inflammatory leucogram and hypoalbuminaemia), present in all dogs on completion of NSAID dosing, resolved within 3 weeks. Resolution of ulceration was confirmed for each dog using the endoscopic procedure previously employed.

Frozen plasma samples were transported under GMP conditions at −80° C. to an analytical facility. Following thawing, the samples were analysed for lactulose concentration using an ion chromatography method previously developed using blank plasma samples spiked with known concentrations of lactulose.

TABLE 1
Ion Chromatography Conditions

| | |
|---|---|
| System | Thermo Scientific ICS 5000 Ion Chromatography System |
| Analytical | Thermo Scientific Dionex CarboPacTM PA 200 |
| Column | BioLCTM 3 × 250 mm Analytical Column, fitted with |
| | a Dionex CarboPacTM PA 200 BioLCTM × 50 mm |
| | Guard column |
| Mobile | Isocratic - 4 mM KOH in Deionised Water (Sigma |
| Phase | Aldrich ≤2 μS/cm) |
| Eluent | EGC 500 KOH Cartridge |
| Generator | |
| Flow Rate; | 500 μL/min; 50 min |
| Run Time | |
| Column | 35° C. |
| Temperature | |
| Compartment | 30° C. |
| Temperature | |
| Injection | 10 μL |
| Volume | |
| Detection | Electrochemical Detector |

Sample preparation for Ion Chromatography analysis: 200 μL aliquots of liquid from each of the plasma samples were diluted to 1000 μL with acetonitrile. The samples were vortexed thoroughly, followed by centrifugation at 15000 rpm for 10 minutes. The supernatants were analysed directly by ion chromatography. Analysis was performed in duplicate.

Calibration Curve: Calibration curves were prepared in the blank plasma sample. Briefly, plasma samples were spiked with varying concentrations of lactulose and were then diluted with acetonitrile to the required final concentration range. The samples were then processed as described above before analysis by ion chromatography. Samples were analysed in duplicate. Linearity was established for plasma lactulose concentrations in the range 5 to 250 μM.

For each dog, the rate and extent of systemic lactulose absorption was increased significantly from baseline following ulcer induction. Peak plasma concentration, area under the concentration-time curve, and the slope of the absorption curve reflected disruption to the gastric mucosal barrier. Comparing individual dogs' plasma concentrations with clinical and endoscopic classification of gastric lesions, rank order was established for disease severity.

TABLE 2
Mean Plasma Lactulose at Baseline and Post Ulcer Induction

| Endoscopic Grade | Plasma Lactulose (μM) | | | | | | | |
|---|---|---|---|---|---|---|---|---|
| of Gastric | 10 min | | 20 min | | 30 min | | 60 min | |
| Ulceration | Mean | CV % | Mean | CV % | Mean | CV % | Mean | CV % |
| Absent (n = 8) | 24.94 | 53% | 39.13 | 55% | 46.04 | 53% | 61.13 | 42% |
| Mild (n = 4) | 44.60 | 59% | 68.67 | 50% | 88.18 | 46% | 110.15 | 37% |
| Moderate to severe | 60.91 | 67% | 94.30 | 39% | 143.85 | 35% | 183.59 | 26% |
| (n = 4) | | | | | | | | |

The results are plotted in FIG. 4.

The plasma lactulose concentration results obtained using the ion chromatography method described above were strongly indicative of the extent and severity of gastric ulceration in each dog following NSAID administration, as assessed using the current gold standard diagnostic test; i.e. endoscopic examination. Following oral administration of a lactulose solution, markedly increased systemic absorption of lactulose was observed in all dogs in the presence of gastric ulceration. Utilising a cut-off value for plasma lactulose concentrations >100 μM within 60 mins of oral administration of a 20% w/v lactulose solution yielded 75% sensitivity and 100% specificity for induced gastric ulceration in the canine study group.

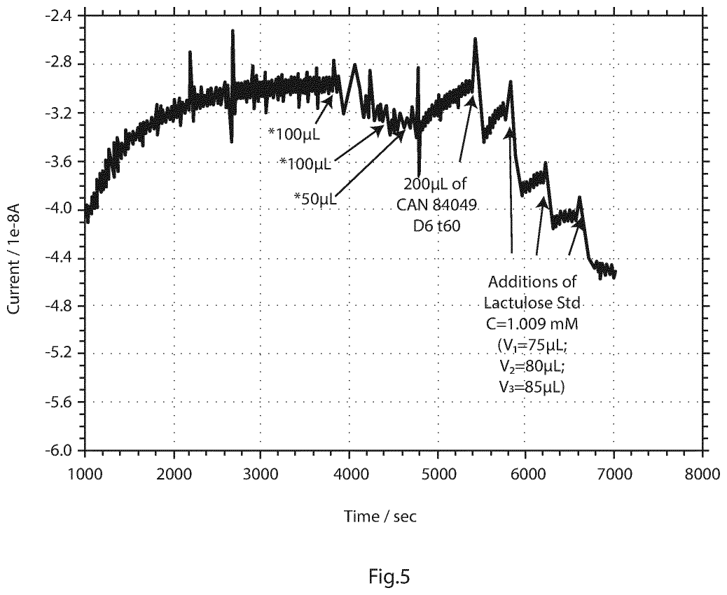

### EXAMPLE 3 (FIG. 5)

An electrochemical biosensor for the indirect detection of lactulose in buffer and plasma samples used an electrocatalytic surface as the working electrode which was modified with a dual enzyme system comprising β-galactosidase (β-Gal) and galactose oxidase (GalOx). The electrochemical biosensor comprised a 3-electrode system of a working electrode (the dual enzyme-modified Pt electrode), an Ag/AgCl reference electrode, and a Pt wire as auxiliary electrode.

The enzymes were immobilised on the surface of the working electrode using a cross-linker. A 2 mm diameter Pt disk was used as the electrocatalytic surface for the working electrode. A 20 μL mix of β-Gal/GalOx/Glutaraldehyde (4 units/13.3 units/0.5 wt. %) was prepared in phosphate-buffered saline (PBS) solution 0.1 M pH 7.3. 5μL of the mix was then drop-cast on the working electrode surface and left to dry at room temperature.

The three electrodes were immersed in 2 mL of PBS solution 0.1 M pH 7.3 at 35° C. to comprise the electrochemical cell and an oxidative potential of +600 mV was applied between the working and reference electrodes for quantification of lactulose in a canine plasma sample by amperometry, as illustrated in FIG. 5. This shows an Amperometric i-t curve for the quantification of lactulose in a canine plasma sample (CAN 84049 D6 t60). In this plot “*” represents additions of blank plasma.

2×100 μL followed by 1×50 μL of blank plasma were added to the electrochemical cell and the system allowed to stabilise between additions.

Following this 200 μL of canine plasma (CAN 84049 D6 t60) was added to the cell and the current response was monitored for a period of 400 s.

Three sequential additions of a volume of 1 mM Lactulose standard (V1=75 μL; V2=80 μL; V3=85 μL) were then added to the cell and the current response was monitored for 400 s after each addition.

A calibration curve was generated based on the standard lactulose additions added to the cell and was used to estimate the lactulose concentration in the original plasma sample to be between 275 to 400 μM.

These results (and also the plots of FIG. 4) indicate that plasma lactulose levels in excess of 100 μM within 60 minutes of oral administration of 0.6 to 0.7 g/kg lactulose in a 20% w/v solution may be used to diagnose gastric ulceration in monogastric animals. More generally, plasma lactulose levels in excess of 100 μM within 60 minutes of oral administration of 0.5 to 1.0 g/kg lactulose indicate the presence of gastric ulceration.

We have surprisingly found that the large lactulose molecule passes much more readily into the bloodstream across ulcerated gastric tissue than through “leaky” tight junctions between intestinal cells as are seen in immunologic disease of the intestinal tract such as coeliac disease. Direct exposure of underlying submucosal tissues in the gastric wall to lactulose facilitates far greater uptake of the molecule than is possible through a porous, but intact mucosal layer. Distinct from absorption reported in other disease conditions, this enhanced uptake occurs soon (<60 mins) after oral administration, reflecting early exposure of the diseased tissue to lactulose, and localising absorption to the upper gastrointestinal tract.

On obtaining such measurements indicating the presence of gastric ulcers in a canine patient, for example, a veterinarian may prescribe therapy in the form of a proton pump inhibitor. Omeprazole extended-release oral capsules are currently a preferred treatment option, and are dosed at 0.5 to 1.0 mg/kg bodyweight, twice daily, for 21 days. Repeat testing of lactulose absorption as described above may be conducted on completion of therapy to ensure resolution of gastric ulceration. Human and equine patients may be similarly treated, but at dose rates of 0.5 and 4 mg/kg respectively, once or twice daily as deemed necessary by the attending clinician. These and other treatments that may be used after detection of gastric ulceration as described herein are outlined in detail below.

Point-of-care lactulose testing represents a simple, economical alternative to gastroscopy for the diagnosis of gastric ulcers. The invention achieves sufficiently low measurement sensitivity in a simple, point-of-care format.

### Gastric Ulceration

The monogastric stomach is an active reservoir that stores, triturates, and slowly dispenses partially digested food into the intestine for further digestion and absorption, and also controls appetite and satiety. Its main secretory function is secretion of gastric acid, which initiates peptic hydrolysis of dietary proteins, liberates vitamin B12 from dietary protein, facilitates duodenal absorption of inorganic iron and calcium, stimulates pancreatic HCO₃— secretion via secretin release, suppresses antral gastrin release, and modulates the intestinal microbiome by killing microorganisms and preventing bacterial overgrowth.

Dogs have lower basal but higher peak gastric acid secretion compared to humans. However, fasting gastric pH is comparable in dogs and humans. In one study the intragastric pH of healthy control dogs remained <2.0 over 85% of the time, with a mean percentage time (MPT) intragastric pH >4.0 of only 4.7% (Tolbert K, Bissett S, King A, et al. Efficacy of oral famotidine and 2 omeprazole formulations for the control of intragastric pH in dogs. J Vet Intern Med. 2011; 25:47-54.). In another study, the median gastric pH was 1.1 and the median percentage of the investigation time that the gastric pH fluctuated between 0.5 and 2.5 was 90.32% (range, 78%-97.4%) (Kook P H, Kempf J, Ruetten M, Reusch C E. Wireless ambulatory esophageal pH monitoring in dogs with clinical signs interpreted as gastroesophageal reflux. J Vet Intern Med. 2014; 28:1716-1723.).

Gastric pH in humans increases with feeding because of the buffering effect of food, but dogs differ because the buffering effect of food is not consistently observed and is much smaller in effect, if present at all. This observation may be caused by higher peak acid output in fed dogs. Another explanation may be differences in methodology, because the pH capsule methodology used in newer studies, unlike digital probes, allows direct adherence to the gastric mucosa and provides direct measurement of intragastric pH.

The horse stomach continuously secretes variable amounts of hydrochloric acid throughout the day and night and secretion of acid occurs without the presence of feed material. Foals secrete gastric acid as early as 2 days of age and acidity of the gastric fluid is high. High acid in the stomach may predispose foals to gastric ulceration. The adult equine stomach secretes approximately 1.5 litres of gastric juice hourly and acid output ranges from 4 to 60 mmoles hydrochloric acid per hour. The pH of gastric contents ranges from 1.5 to 7.0, depending on region measured. A near neutral pH can be found in the dorsal portion of the esophageal region near the lower oesophageal sphincter, whereas more acidic pHs can be found in the glandular region near the pylorus (1.5-4.0). Gastric emptying of a liquid meal occurs within 30 minutes, whereas complete gastric emptying of a roughage hay meal occurs in 24 hours.

### Gastric Mucosal Barrier

The gastric mucosal barrier is a complex defence mechanism, protecting the normal mucosa from the harsh chemical environment of the gastric luminal contents. Gastric luminal peptides and gastric distention provide strong stimulation for gastric acid production. In response to stimulation, parietal cell H+/K+-ATPase and KCl transporters become incorporated into the parietal cell canalicular membrane. Hydrogen ions are released into the gastric lumen from parietal cells upon stimulation in exchange for potassium, resulting in a very acidic environment.

The gastric mucosal barrier protects the gastric epithelium from the highly acidic luminal contents. Tight junctions seal the cellular layers of the gastric mucosa, ensuring that the luminal contents do not leak into or around these cells. A thick, bicarbonate-rich mucous layer covers the epithelial surface. The small amount of gastric acid that diffuses into epithelial cells is quickly cleared by the high blood flow to this area. This high blood flow also supports cellular metabolism and rapid renewal of injured cells. Local production of prostaglandins E2 and I2 help maintain the GI mucosal blood flow and integrity, increase mucous and bicarbonate secretion, decrease acid secretion, and stimulate epithelial cell turnover.

In the normal GI tract, the potential disruptive properties of the luminal contents are balanced by the defence mechanisms of the GI mucosal barrier. However, many drugs and diseases have the potential to upset the balance between the harsh luminal contents and the GI protective barrier. GI ulceration primarily targets the stomach and/or duodenum.

A defect in the normal GI mucosal barrier leads to a self-perpetuating cycle of further mucosal damage. Injury to this barrier allows hydrochloric acid, bile acids, and proteolytic enzymes to degrade the epithelial cells, disrupt lipid membranes, and induce inflammation and apoptosis. Back diffusion of luminal contents through the tight junctions leads to inflammation and haemorrhage of the GI cells, with further acid secretion mediated by inflammatory cells and their products. Mast cell degranulation occurs, causing histamine release that perpetuates further gastric acid secretion. The inflammatory environment also causes decreased blood flow, resulting in ischemia, decreased ability for cellular repair, and reduced secretion of mucus and cytoprotective prostaglandins. Mucosal ulceration can result, exposing the submucosa or deeper layers of the GI tissue to the harsh chemical luminal contents.

### Aetiology of Gastric Ulceration in Different Species

### Horses

Ulcers in the non-glandular squamous mucosa are associated with repeated direct insult from ultra-low pH fluid normally found in the glandular region of the stomach in horses. Pressure increases inside the abdomen (associated with exercise), collapsing the stomach and forcing the acid gastric contents upward. The more fluid (and highly acidic) contents of the lower stomach come in contact with the non-glandular squamous mucosa, causing inflammation and, potentially, erosions to varying degrees.

The causes of ulcers in the glandular mucosa of the stomach are less well defined. Use of nonselective NSAIDs are known to reduce blood flow to the GI tract, causing decreased production of the muco-bicarbonate matrix by the gastric glandular mucosa and resulting in ulceration. This is not a consistent finding, however. Additionally, attempts have been made to isolate and/or correlate evidence of Helicobacter organisms from the stomach of horses with and without gastritis and ulcers. Results of these studies have been equivocal or negative, and the role of this organism in glandular equine gastric ulcers has not been determined.

### Humans

Helicobacter pylori and NSAIDs disrupt normal mucosal defence and repair, making the mucosa more susceptible to acid. H. pylori infection is present in 50 to 70% of patients with duodenal ulcers and in 30 to 50% of patients with gastric ulcers. If H. pylori is eradicated, only 10% of patients have recurrence of peptic ulcer disease, compared with 70% recurrence in patients treated with acid suppression alone. NSAIDs now account for >50% of peptic ulcers.

Cigarette smoking is a risk factor for the development of ulcers and their complications. Also, smoking impairs ulcer healing and increases the incidence of recurrence. Risk correlates with the number of cigarettes smoked per day. Although alcohol is a strong promoter of acid secretion, no definitive data link moderate amounts of alcohol to the development or delayed healing of ulcers. Very few patients have hypersecretion of gastrin caused by a gastrinoma (Zollinger-Ellison syndrome).

### Dogs

In dogs, NSAID administration, neoplasia, and hepatic disease are the most common reported causes of gastric ulceration. NSAIDs can cause direct topical damage to the GI mucosa, and inhibition of cyclooxygenase (COX)-1 decreases production of protective prostaglandins. The use of COX-2-specific NSAIDs is thought to decrease GI ulceration, but ulceration and perforation can still occur with use of these medications.

Primary GI neoplasia such as lymphoma, adenocarcinoma, leiomyoma, and leiomyosarcoma can result in ulceration due to local effects of the tumour. Additionally, paraneoplastic syndromes secondary to mast cell tumours and gastrinomas (Zollinger-Ellison syndrome) have been associated with increased gastric hydrochloric acid production and subsequent ulceration in dogs.

Various hepatic diseases (e.g., acute hepatic injury, intrahepatic portosystemic shunt) are associated with gastroduodenal ulceration, but the mechanism of disease is not known. Possible causes include increased gastric acid secretion and alterations in mucosal blood flow, potentially leading to ulcer formation.

Other causes of ulceration in dogs include major trauma, spinal disease, renal disease, hypoadrenocorticism, GI inflammation such as inflammatory bowel disease or presence of a traumatic foreign body, systemic inflammation such as pancreatitis and sepsis, and extreme exercise such as sled dog racing. Combining NSAID and corticosteroid therapy will increase risk of GI ulceration and is contraindicated.

### Treatment of Gastric Ulceration

Treatment of gastric ulceration follows a common approach across monogastric species.

### Elimination of Predisposing Lifestyle, Dietary and Stress Factors

In humans, lifestyle changes such as cessation of smoking and reducing or managing stress may aid in resolution of gastric ulceration and prevention of recurrence. In horses, a break from training and alterations to the diet to increase the amount of roughage consumed may similarly aid recovery.

### Elimination of Causative Medications

NSAIDs are a common cause of gastric ulceration in all treated monogastric species. A recent study has shown that 75% of dogs receiving chronic NSAID therapy have overt or silent gastric ulceration, while approximately 12% of human patients likewise develop gastric lesions within the first 12 weeks of NSAID therapy. In asymptomatic cases, NSAIDs may justifiably be continued in the interest of managing an underlying inflammatory or painful disorder, but administration should cease in human, equine, canine and other patients presenting with symptoms of ulceration. Dysbiosis that occurs as a result of therapeutic gastric pH suppression may potentiate the damaging effects of NSAIDs on gastric and intestinal mucosa, and so co-administration of gastric acid suppressants and NSAIDs should be avoided. There is conflicting evidence as to the ulcerogenic potential of corticosteroids when used as monotherapy in all species, but NSAID—corticosteroid combination therapy has potent effects in weakening the gastric mucosal barrier and ulcer development.

### Surgery

Surgical excision of deep and/or perforated gastric ulcers is sometimes performed in all species, with primary repair of the gastric wall by apposing healthy tissue beyond the ulcer margins. However, due to high perioperative morbidity and mortality the procedure is carried out only by necessity and is best avoided through early intervention and aggressive medical therapy. When gastric ulceration occurs due to a neoplastic process such as gastrinoma, surgical excision of the tumour may be curative.

### Antisecretory Medications

Reduction of acid secretion and the resultant increase of gastric pH are fundamental to successful treatment of gastric ulceration. In humans, healing of gastric ulcers is highly correlated with the degree of gastric acid suppression. For people with ulcers, optimal treatment involves maintaining an intragastric pH ≥3 for 18 to 20 hours a day (i.e., approx. 75% of the day) and little benefit is associated with more extensive gastric acid suppression. Intragastric pH >6 is necessary to achieve haemostasis with acute gastrointestinal tract bleeding, as pH values >6 allow adequate platelet aggregation and prevent dissolution of blood clots.

The optimal degree of gastric acid suppression necessary for treating acid-related diseases in other monogastric species has not been established, and critical intragastric pH thresholds in dogs and horses may differ from those established for humans because of differences in gastric physiology. Regardless, it seems likely that the degree and duration of gastric acid suppression will correlate with ulcer healing in all monogastric species.

### Antacids

Antacids are the oldest of the gastrointestinal (GI) protectants and comprise a group of inorganic, relatively insoluble salts of aluminium hydroxide (Al(OH)₃), calcium carbonate (CaCO₃), and magnesium hydroxide (Mg(OH)₂) that lack systemic effects. Antacids may be beneficial by decreasing pepsin activity, binding to bile acids in the stomach, and stimulating local prostaglandin synthesis. Antacids have historically been used in humans and dogs but are ineffective in the treatment of gastric ulceration. Constipation is the most common adverse effect in both species.

### Histamine Type-2 Receptor Antagonists

Histamine type-2 receptor antagonists (H2RAs; e.g., cimetidine, ranitidine, and famotidine) inhibit acid secretion by competitively blocking H-2 receptors on the parietal cell, thus decreasing gastric acid secretion. Continuous H2RA administration results in pharmacological tolerance within days, which can be demonstrated through gastric pH monitoring. Tolerance occurs even more rapidly (12-72 hours) in human subjects when famotidine is administered intravenously. Because of this tolerance, abrupt discontinuation of H2RAs causes rebound acid hypersecretion in humans as a result of the trophic properties of gastrin on enterochromaffin like cells. This phenomenon has not yet been documented in dogs or horses, although it appears reasonable to assume its occurrence. The H2RAs have been shown to increase gastric pH in all monogastric species evaluated to date, though appear to be less effective than proton pump inhibitors (PPIs).

### Proton Pump Inhibitors

The PPIs (e.g. omeprazole, pantoprazole, esomeprazole, and lansoprazole) are substituted benzimidazole drugs that target the final common pathway of acid production. The PPIs are significantly more effective than H2RAs in increasing gastric pH and preventing and healing gastric ulceration. The PPIs irreversibly inactivate the acid secretory pathway, resulting in a prolonged effect after administration. Maximal inhibitory effect is achieved within approximately 2-4 days of PPI administration. With repeated dosing, omeprazole may reduce its own metabolism, increasing its effectiveness through inhibition of cytochrome P450 enzymes. While several PPIs (e.g. omeprazole, esomeprazole, lansoprazole, pantoprazole) have been approved for use in human gastric ulcer patients, only omeprazole has received regulatory approval for use in horses, and no PPI has been approved for use in dogs or other monogastrics to date. It has been consistently shown that PPIs are superior to H2RAs for increasing intragastric pH and facilitating gastric ulcer healing. For all monogastric species, it has been suggested that PPIs should be gradually tapered after administration for ≥4 weeks to avoid rebound gastric acid hypersecretion.

### Sucralfate

Sucralfate (Carafate) is a complex salt of sucrose octasulphate and aluminium hydroxide that may be safely used in the treatment of gastric ulceration in humans, dogs, and horses. Its mechanism of action in acid-peptic disease is multifactorial. Sucralfate forms stable complexes with protein in damaged mucosa where there is a high concentration of protein, either from fibrinogen, albumin, or globulins from the exudate of an ulcer or from damaged cells. In an acidic environment, sucralfate becomes viscous and partially dissociates into sucrose sulphate and aluminium hydroxide. The sucrose sulphate moiety is an anion and binds electrostatically with the positively charged proteins in the damaged mucosa. Sucralfate interferes with the action of pepsin either by preventing pepsin digestion of protein substrates, by binding to pepsin, or by providing a barrier to prevent diffusion of pepsin. In addition, the protection afforded by sucralfate against oesophageal acid injury is mediated by intraluminal pH buffering via aluminium hydroxide and protection against H+ entry and injury via sucrose octasulphate. Sucralfate also may provide a barrier for bile salts. Sucralfate is known to stimulate prostaglandin production in the gastric epithelium. This may be a potential secondary effect of sucralfate in the oesophagus, although the importance and effectiveness of sucralfate as an agent for the treatment of erosive esophagitis is not as established as it has been for H2RAs or PPIs. In foals, sucralfate had a protective effect on gastric ulcers associated with intravenous administration of high-dose NSAIDs.

### Combination Therapy for Helicobacter

In humans, H. pylori is implicated in the majority of cases of gastric ulceration. While non-H. pylori Helicobacter infection has been reported in other monogastric species, the significance of these infections and the clinical benefit of eliminating them remains questionable. Anti-H. pylori therapy in humans currently consists of multiple drugs, either simultaneously or sequentially, and PPIs are almost always an integral component of treatment.

While choosing a treatment regimen for H. pylori, human patients should be asked about previous antibiotic exposure and this information should be incorporated into the decision-making process. For first-line treatment, clarithromycin triple therapy should be confined to patients with no previous history of macrolide exposure who reside in areas where clarithromycin resistance amongst H. pylori isolates is known to be low. Most patients are better served by first-line treatment with a PPI, bismuth subcitrate, tetracycline and metronidazole (bismuth quadruple therapy) or a PPI, clarithromycin, amoxicillin, and metronidazole for 10 to 14 days. When first-line therapy fails, a salvage regimen should avoid antibiotics that were previously used. If a patient received a first-line treatment containing clarithromycin, bismuth quadruple therapy or salvage regimens that include levofloxacin are the preferred treatment options. If a patient received first-line bismuth quadruple therapy, clarithromycin or levofloxacin-containing salvage regimens are the preferred treatment options. The 3-year recurrence rate for gastric ulcers in humans is <10% when H. pylori is successfully eradicated but is >50% when it is not. Thus, a patient with recurrent disease should be tested for H. pylori and treated again if the tests are positive.

The references mentioned in this specification are herein incorporated by reference in their entirety.

The invention is not limited to the embodiments hereinbefore described, which may be varied in construction and detail.

1. A method for determining the concentration of lactulose in a blood sample, the method comprising the steps of:
a) converting lactulose in the blood sample into galactose;
b) converting the galactose into D-galacto-hexodialdose and hydrogen peroxide in the presence of oxygen or a redox mediator;
c) detecting one or more of the following which are provided by step (b):
a quantity of hydrogen peroxide,
a quantity of D-galacto-hexodialdose,
a change in the quantity of redox mediator, or
a change in the quantity of oxygen; and
d) processing the detected quantity or change in quantity provided by step (c) to generate an output which is representative of the concentration of lactulose in the blood sample.

2. A method as claimed in claim 1, wherein at least one of the step (a) conversion of lactulose in the blood sample into galactose or step (b) conversion of galactose into D-galacto-hexodialdose and hydrogen peroxide is an enzymatic conversion.

3. A method as claimed in claim 2, wherein the enzyme for converting lactulose in the blood sample into galactose is β-galactosidase.

4. (canceled)

5. A method as claimed in claim 2, wherein the step (b) enzyme for converting the galactose into D-galacto-hexodialdose and hydrogen peroxide is galactose oxidase.

6. (canceled)

7. A method as claimed in claim 2, wherein the step (a) and/or step (b) enzymatic conversions are performed with an electrochemical biosensor by contact with at least one enzyme immobilized on a working electrode surface of the biosensor when the working electrode is exposed to the blood sample and a voltage is applied across the working electrode and a reference electrode of the biosensor.

8. A method as claimed in claim 7, wherein the working electrode includes Pt and the at least one enzyme is drop cast onto the working electrode surface using a cross-linker.

9. (canceled)

10. A method as claimed in claim 7, wherein the at least one enzyme includes are at least two enzymes including β-galactosidase for the step (a) enzymatic conversion and galactose oxidase for the step (b) conversion.

11. (canceled)

12. A method as claimed in claim 7, wherein the hydrogen peroxide is detected in step (c) by detection of current flow between the electrodes.

13. (canceled)

14. A method as claimed in claim 1, wherein step b) includes converting the galactose into D-galacto-hexodialdose and hydrogen peroxide in the presence of a redox mediator, and the redox mediator comprises one or more selected from ferrocene and its derivatives, quinone-based compounds, ferrocyanide, ferricyanide, redox organic polymers, or inorganic complexes or combinations thereof.

15. A method as claimed in claim 1, wherein the blood sample is a sample of blood plasma.

16. A method as claimed in claim 1, wherein the blood sample is from a human or a non-human animal.

17-19. (canceled)

20. An apparatus for determining the concentration of lactulose in a blood sample, the apparatus comprising:
a converter comprising a substrate for:
converting lactulose in the blood sample into an entity including galactose; and
converting the galactose into D-galacto-hexodialdose and hydrogen peroxide in the presence of oxygen or a redox mediator;
a detector for detecting:
one or more of the following provided by the converter:
quantity of hydrogen peroxide,
quantity of D-galacto-hexodialdose,
a change in the quantity of redox mediator, or
a change in the quantity of oxygen; and
a processor for:
processing the detected quantity or change in quantity provided by the detector to generate an output which is representative of the concentration of lactulose in the blood sample.

21-25. (canceled)

26. An apparatus as claimed in claim 20, wherein the converter and the detector are integrated in an electrochemical biosensor including a working electrode and a reference electrode, at least one enzyme is immobilized on a surface of the working electrode and the apparatus includes a driver to apply a voltage across the electrodes.

27-30. (canceled)

31. A method for diagnosing gastric ulceration in a subject, the method comprising the steps of:
administering lactulose to the subject;
taking a sample of blood from the subject after administering lactulose;
converting lactulose in the blood sample into galactose;
converting the galactose into D-galacto-hexodialdose and hydrogen peroxide in the presence of oxygen or a redox mediator;
detecting one or more of the following after converting the galactose into D-galacto-hexodialdose and hydrogen peroxide:
a quantity of hydrogen peroxide,
a quantity of D-galacto-hexodialdose,
a change in the quantity of redox mediator, or
a change in the quantity of oxygen; and
in vitro determining the concentration of lactulose in the blood sample as an indicator of gastric ulceration.

32. A method as claimed in claim 31, further comprising the step of converting the blood sample into plasma after taking the blood sample, and determining the concentration of lactulose in the plasma; wherein a concentration of more than 100 micromolar lactulose in the plasma within 60 minutes of administration of the lactulose is indicative of gastric ulceration.

33. (canceled)

34. A method as claimed in claim 31, wherein the lactulose is administered in an amount of from 0.5 to 1.0 gram per kg bodyweight of the subject.

35. (canceled)

36. A method as claimed in claim 31, wherein taking a blood sample occurs between 5 minutes and 100 minutes after administering the lactulose.

37-40. (canceled)

41. A method as claimed in claim 31, wherein each of the conversion of lactulose in the blood sample into galactose and the conversion of galactose into D-galacto-hexodialdose and hydrogen peroxide is an enzymatic conversion.

42. A method as claimed in claim 41, wherein the enzyme for converting lactulose in the blood sample into galactose is β-galactosidase, and the enzyme for converting the galactose into D-galacto-hexodialdose and hydrogen peroxide is galactose oxidase.

43-45. (canceled)

46. A method as claimed in claim 41, wherein at least one of the conversion of lactulose in the blood sample into galactose or the conversion of galactose into D-galacto-hexodialdose and hydrogen peroxide is performed with an electrochemical biosensor by contact with at least one enzyme immobilized on a working electrode surface when the working electrode is exposed to the blood sample and a voltage is applied across the working electrode and a reference electrode of the biosensor.

47-48. (canceled)

49. A method as claimed in claim 46, wherein the at least one enzyme includes at least two immobilized enzymes including β-galactosidase and galactose oxidase.

50-53. (canceled)

54. A method as claimed in claim 31, wherein the blood sample is from a human or a non-human animal.

55-57. (canceled)
